(19) United States
(12) Patent Application Publication
(10) Pub. No.: US 2024/0358596 A1
(43) Pub. Date: Oct. 31, 2024

(54) SYSTEMS AND METHODS FOR MANAGING MEDICINE USAGE

(71) Applicant: Bradley Scott Ingham, Brazoria, TX (US)

(72) Inventor: Bradley Scott Ingham, Brazoria, TX (US)

(21) Appl. No.: 18/644,813

(22) Filed: Apr. 24, 2024

Related U.S. Application Data
(60) Provisional application No. 63/462,826, filed on Apr. 28, 2023.

(51) Int. Cl. A61J 7/02; A61J 7/04

(52) U.S. Cl. CPC A61J 7/02; A61J 7/0409; A61J 2200/30; A61J 2200/70

(57) ABSTRACT

Systems and methods are provided for managing medicine usage. The system includes a medicine management apparatus that is configured to receive a medicine container. The medicine management apparatus tracks usage of medicine in the medicine container by monitoring when the medicine container is removed from the medicine management apparatus. The medicine management apparatus also provides alerts to a user when it is time for the user to take another dosage of medicine and alerts the user when the amount of medicine in the medicine container is low.

### CROSS-REFERENCE TO RELATED APPLICATION(S)

This application claims the benefit of U.S. Provisional Application No. 63/462,826, filed Apr. 28, 2023, the disclosure of which is incorporated herein by reference as if set forth in full.

### BACKGROUND

Not taking medication as prescribed can have detrimental results. Additionally, transferring medication to a different container than the originally-provided prescription container may result in mixing with other types of medication and may also result to injury to a user of the medication.

### BRIEF DESCRIPTION OF THE DRAWINGS

The detailed description is set forth with reference to the accompanying drawings. The drawings are provided for purposes of illustration only and merely depict example embodiments of the disclosure. The drawings are provided to facilitate understanding of the disclosure and shall not be deemed to limit the breadth, scope, or applicability of the disclosure. In the drawings, the left-most digit(s) of a reference numeral may identify the drawing in which the reference numeral first appears. The use of the same reference numerals indicates similar, but not necessarily the same or identical components. However, different reference numerals may be used to identify similar components as well. Various embodiments may utilize elements or components other than those illustrated in the drawings, and some elements and/or components may not be present in various embodiments. The use of singular terminology to describe a component or element may depending on the context, encompass a plural number of such components or elements and vice versa.

FIG. 1 illustrates an apparatus for managing medicine usage, in accordance with one or more example embodiments of the disclosure.

FIGS. 2A-2B illustrate an example enclosure of the apparatus of FIG. 1, in accordance with one or more example embodiments of the disclosure.

FIGS. 3A-3B illustrate an example of a use case for managing medicine usage, in accordance with one or more example embodiments of the disclosure.

FIG. 4 illustrates a method for managing medicine usage, in accordance with one or more example embodiments of the disclosure.

FIG. 5 illustrates an example system for managing medicine usage, in accordance with one or more example embodiments of the disclosure.

FIG. 6 illustrates a computing device, in accordance with one or more example embodiments of the disclosure.

### DETAILED DESCRIPTION

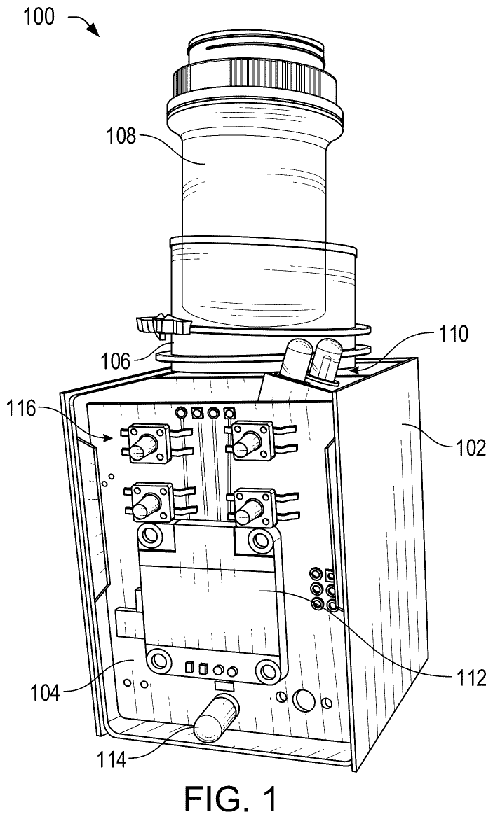

This disclosure relates to, among other things, systems and methods for managing medicine usage. Particularly, a medicine management apparatus (an example of such a medicine management apparatus is shown in FIG. 1) is described herein that is used to track and facilitate the usage of different types of medicine. The medicine management apparatus may be configured to hold a medicine container (such as a prescription pill bottle or other type of container for medicine in any form, such as liquid, solid, inhalant, etc.). The medicine management apparatus may also include a controller with processing capabilities, one or more sensors, and a display. The medicine management apparatus and associated system ensures that medicine is kept within a prescription medicine container to prevent mixing of certain medicine with other types of medicines. The medicine management apparatus also allows for a user to track usage of the medicine(s) to ensure that the proper dosages are taken at the proper intervals, and also to determined when the amount of remaining medicine in the medicine container is low.

The medicine management apparatus may include a controller with processing capabilities to facilitate tracking the amount of medicine remaining in the medicine container. The controller may receive data from the one or more sensors. Using this data, the controller may determine when the medicine container has been removed from the medicine management apparatus by a user (such as when the user removes the medicine container to take a dosage of medicine from the medicine container). For example, the one or more sensors may be pressure sensors and the controller may determine that the medicine container has been removed when the pressure data falls below a threshold value. As another example, the one or more sensors may be optical sensors, proximity sensors, and/or any other types of sensors used individually or in combination.

When the controller determines that the medicine container has been removed from the medicine management apparatus, the controller may decrement a counter. Alternatively, the medicine management apparatus may decrement the counter when it is determined that the medicine container has been returned to the medicine management apparatus, or when it has been determined that the medicine container has been removed from and returned to the medicine management apparatus.

The counter may be a value (such as a numerical value, for example) representing the amount of medication remaining in the medicine container (as a non-limiting example, a counter indicating a number of pills remaining in the medicine container). That is, when the medicine container is removed from the medicine management apparatus, the controller may assume that a single dosage of the medication has been removed from the medicine container and taken by the user. For example, if the counter initially indicates that there are five pills remaining and the dosage is one pill, the counter may be decremented by one when the medicine container is determined to be removed from 9 and/or returned to) the medicine management apparatus. There may also exist mechanisms by which it may be verified that the exact dosage amount was removed from the medicine container as well (these mechanisms are described in further detail with respect to FIG. 1).

In addition to tracking the amount of medication remaining in the medicine container, the medicine management apparatus may also serve other functions associated with the management of the medicine usage. As another function, the medicine management apparatus may produce alerts based on various types of conditions. For example, the medicine management apparatus may produce an alert when a pre-determined amount of time has passed since the user last took a dosage of medicine (indicating that it is time for the user to take another dosage of medicine). The alert may be a visual alert, such as a flashing light emitting diode (LED), an auditory alert, such as a “chirp” or other sound produced by a speaker, a combination of the two, or any other types of alerts. In some instances, the alert may be produced until it is determined that the medicine has been taken by the user (or may only be produced once or continuously for only a fixed period of time). As another example, the medicine management apparatus may produce an alert when the controller determines that an improper dosage of medicine was removed from the medicine management apparatus. As yet another example, the medicine management apparatus may produce an alert when the medicine container has not been returned to the medicine management apparatus after having been removed from the medicine management apparatus.

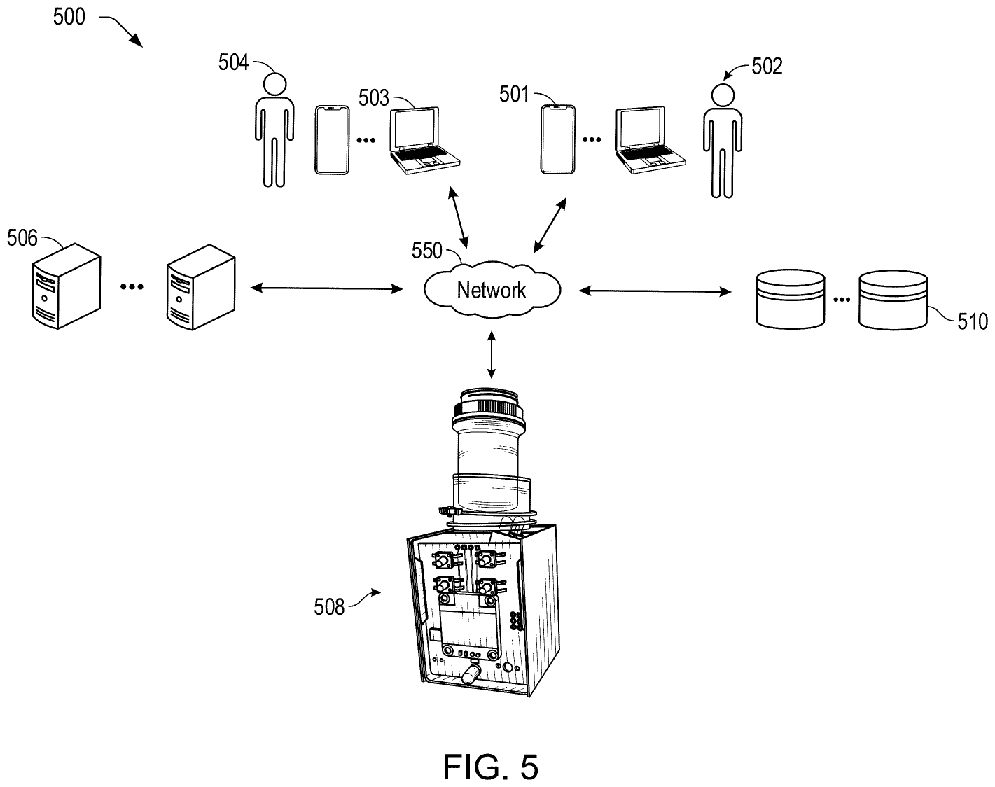

The medicine management apparatus may be a single device included within an overall system used to manage usage of medicine (an example of such as a system is shown in FIG. 5). The medicine management apparatus may also be in communication with user devices, such as smartphones or other types of devices. For example, a user may access an application associated with the medicine management apparatus via the user device to view information produced by the medicine management apparatus (e.g., amount of medicine remaining in a medicine container, any alerts produced by the medicine management apparatus, etc.).

In some instances, multiple medicine management apparatuses may also be used or a single medicine management apparatus capable of holding and/or tracking multiple different types of medicines and/or medicine containers may also be used. For example, a single medicine management apparatus may include compartments for holding different medicine containers such that the different types of medicines associated with the different medicine containers may be individually tracked by the medicine management apparatus in a similar manner that a single type of medication may be tracked. Alternatively, or additionally, an individual medicine management apparatus may be used for each individual medicine container. In such cases, the individual medicine management apparatuses may be in communication with one another through any suitable wired or wireless communication protocols (and/or in communication with any other devices in the system).

Turning to the figures, FIG. 1 illustrates a medicine management apparatus 100. In one or more embodiments, the medicine management apparatus 100 includes an enclosure 102 housing a controller 104. Mounted to the enclosure 102 may be a holding compartment 106 configured to receive and hold a medicine container 108 (or other type of container for holding medicine, which may be generally referred to herein as a “medicine container”). The holding compartment 106 may be at least partially open (for example, the holding compartment 106 may include an opening at a top portion of the holding compartment 106 as shown in FIG. 1). In some embodiments, the holding compartment 106 may also be completely enclose the medicine container 108 once the medicine container 108 is provided in the holding compartment 106.

The medicine container 108 may be any type of container that may hold any number of different types of medicines in various forms. For example, the medicine container 108 may be a prescription pill bottle including medicine in pill form. The medicine container 108 may also hold medicine in a liquid form or in any other form as well. In certain embodiments, the medicine may also be provided directly within the holding compartment 106 as well.

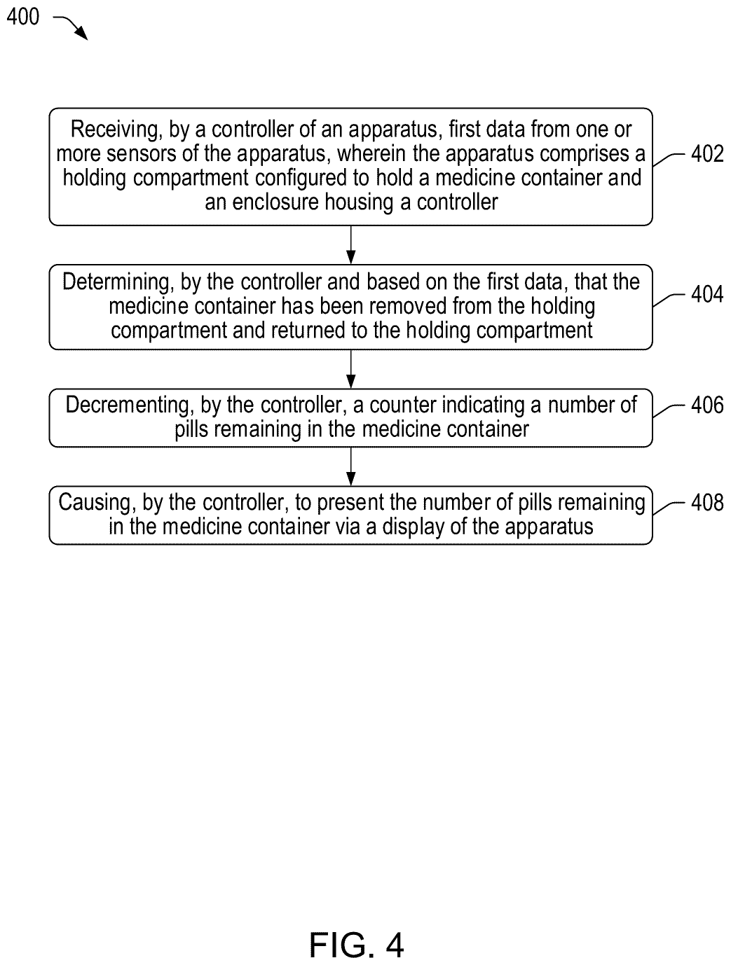

The controller 104 may provide functionality associated with managing the usage of the medicine within the medicine container 108. For example, the controller 104 may be a printed circuit board (PCB) that includes a processor configured to implement logic to facilitate proper usage of the medicine in the medicine container 108 (for example, providing alerts when the medicine needs to be taken, tracking the number of pills that are taken at a time, tracking the number of pills remaining in the medicine container 108, etc.). Additional details about the operations performed by the controller 104 are provided with respect to at least FIG. 4.

To facilitate the management of the medicine usage, one or more sensors 110 may be in electrical communication with the controller 104. For example, the one or more sensors 110 may be provided directly on the PCB or may be separate from the PCB. The one or more sensors 110 may be used to track the position of the medicine container 108 relative to the medicine management apparatus 100, thereby allowing the controller 104 to track the usage of the medicine. For example, when data from the one or more sensors 110 indicates that the medicine container 108 has been removed from the holding compartment 106 and returned back into the holding compartment 106 (or only removed from or returned to the holding compartment 106, etc.), then the controller 104 may determine that a dosage of medicine from the medicine container has been used.

The one or more sensors may be any number of different types of sensors capable of producing data used to make this determination. For example, the one or more sensors 110 may be pressure sensors used to detect when a pressure decreases (e.g., the medicine container 108 has been removed from the holding compartment 106) or increases (e.g., the medicine container 108 has been returned to the holding compartment 106). As another example, the one or more sensors 110 may be optical sensors used to visually detect the position of the pull bottle 108 relative to the holding compartment 106). As yet another example, the one or more sensors 110 may be proximity sensors. Additionally, in some instances, combinations of different types of sensors may also be used (for example, for redundancy purposes).

The data from the one or more sensors 110 may also be used to verify that the correct dosage of medicine was taken when the medicine container 108 was removed from the holding compartment 106. For example, the controller 104 may have stored information (for example, stored in local memory) about the weight of individual pills of the medicine included in the medicine container 108. The controller 104 may also have stored information about the number of pills in a recommended dosage of the medicine. This information may also be obtained by the controller 104 from a remote system (such as a remote server, etc.) via any known wired or wireless communication protocols. Thus, when the medicine container 108 is returned to the holding compartment 106, the difference in a first pressure determined while the medicine container 108 is removed from the holding compartment 106 and a second pressure determined when the medicine container 108 is returned to the holding compartment 106 may be compared to a corresponding weight of a dosage of the medicine. If the result to the comparison is a value that is less than or greater than a threshold value (to account for error), then it may be determined that an improper dosage was taken. This determination may also be made in any other suitable manner using any other types of sensors.

The controller may also include one or more input/output (I/O) components. For example, the input components may include one or more buttons, switches, keys, and/or any other types of input components that allow a user to input information to the controller 104 and/or control certain functionality of the controller 104. For example, the input components may be used to cycle through the display presenting different types of information. For example the user may press a button to cause the display to present an amount of medicine remaining in the medicine container 108 and may press the button again (or another button) to cause the display to present a number of days remaining that the medicine will last based on the amount of remaining medicine, the dosage amount, and the frequency at which the medicine is prescribed to be taken.

As another example, the input components may allow the user to manually initialize the medicine management apparatus for a particular medicine. When the medicine is initially introduced to the medicine management apparatus for tracking, the user may manually input relevant information, such as the type of medicine, the amount of medicine currently in the medicine container 108, the frequency at which the medicine is prescribed to be taken, the prescribed dosage, and/or any other types of relevant information. Some or all of this information may also be automatically be obtained from data stored in local memory or data obtained from a remote system, such as a remote server. For example, a user may indicate a type of medicine and the medicine management apparatus may obtain the remaining information from an external source.

The input components may also allow a user to configure operational parameters of the apparatus. For example, the user may be able to input type of alerts that should be produced, the conditions that trigger alerts to be produced, whether alerts should be transmitted to another device, such as a smartphone of a user, etc.

In addition to providing this information to the controller 104 via the input components, the user may also be able to provide similar types of information through a separate device. For example, an application associated with the medicine management apparatus may be installed on a user device, such as a smartphone, laptop computer, desktop computer, etc. The user may provide inputs to the application and the user device may transmit instructions to the medicine management apparatus based on the inputs to the application.

The output components provide information to the user. For example, the output components may include a display 112. The display 112 may be any type of display, such as an liquid crystal display (LCD), light emitting diode (LED) display, quantum dot light emitting diode (QLED) display, organic light emitting diode (OLED) display, etc. The display 112 may present any information relevant to the management of the usage of the medicine. For example, the display 112 may present a current number of pills remaining in the medicine container 108 and any other types of relevant information. The output components may also include other types of components capable of providing an alert to the user, such as one or more speakers for producing an auditory alert, etc.

The output components may also include other components capable of providing visual information, such as an LED 114. The LED 114 may be used to provide alerts along with, or alternatively to, the display 112. For example, the LED 114 may illuminate when it is time for a user to take another dosage of the medicine in the medicine container 108. The output components may also be components capable of producing auditory information, such as a speaker (not shown in the figure). For example, an auditory alert may also be produced when it is time for the user to take a dosage of medication. Any other types of output components may also be used.

Any of the different types of output components may be used simultaneously or individually. For example, an alert indicating that the user should take a dosage of medicine may include an illumination of the LED 114 and an auditory alert produced through the speaker. Alternatively, the alert may only be a visual alert or only an auditory alert. The user may also configure the types of output components that are used for alerts via the input components of the controller 104.

The medicine management apparatus 100 may also include a battery used to power the controller 104 (for example, the battery may be provided on the printed circuit board of the controller 104 or may be a standalone battery). Alternatively, the medicine management apparatus 100 may be configured to receive power from.

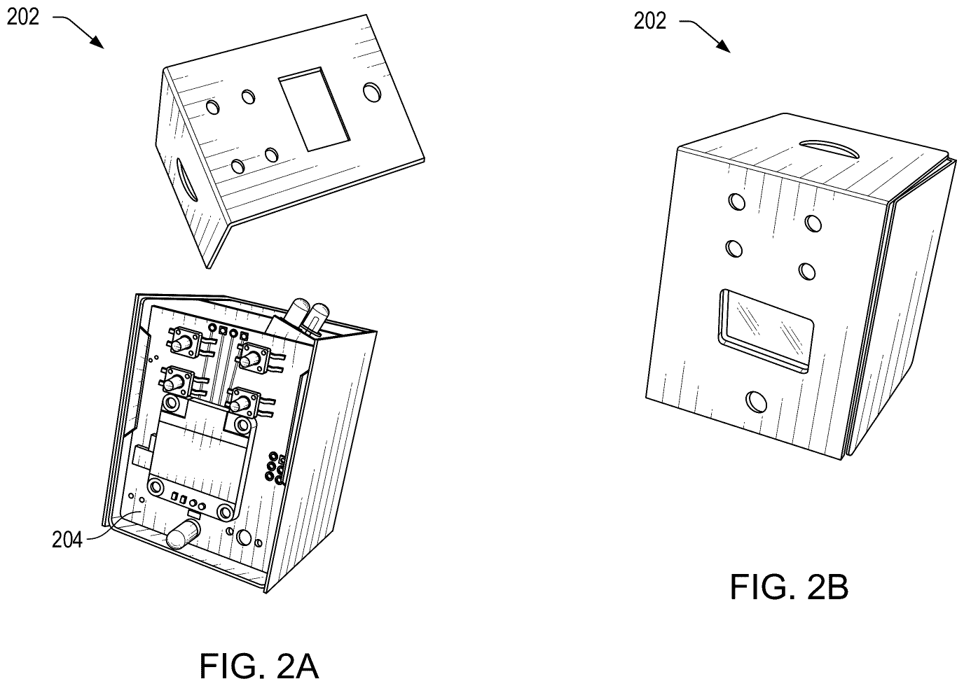

FIGS. 2A-2B provide another illustration of an enclosure 202 and controller 204 included within the enclosure 202. The enclosure 202 and controller 204 may be the same as, or similar to the enclosure 102 and controller 104. The enclosure 202 may be formed through any suitable methods, such as injection molding, etc. The enclosure may be made from any material, such as a plastic, metal, etc. FIG. 2A shows that the enclosure 202 may be made from multiple components that are combined together to form a single enclosure. The different components of the enclosure may be separated such that the controller 104 may be provided within at least one of the components of the enclosure 202. The components may then be combined to form the single enclosure 202 with the controller 204 included within the enclosure 202. The enclosure 202 may also include one or more apertures through which different components of the controller 204 may be accessed (for example, I/O elements, sensors, etc.).

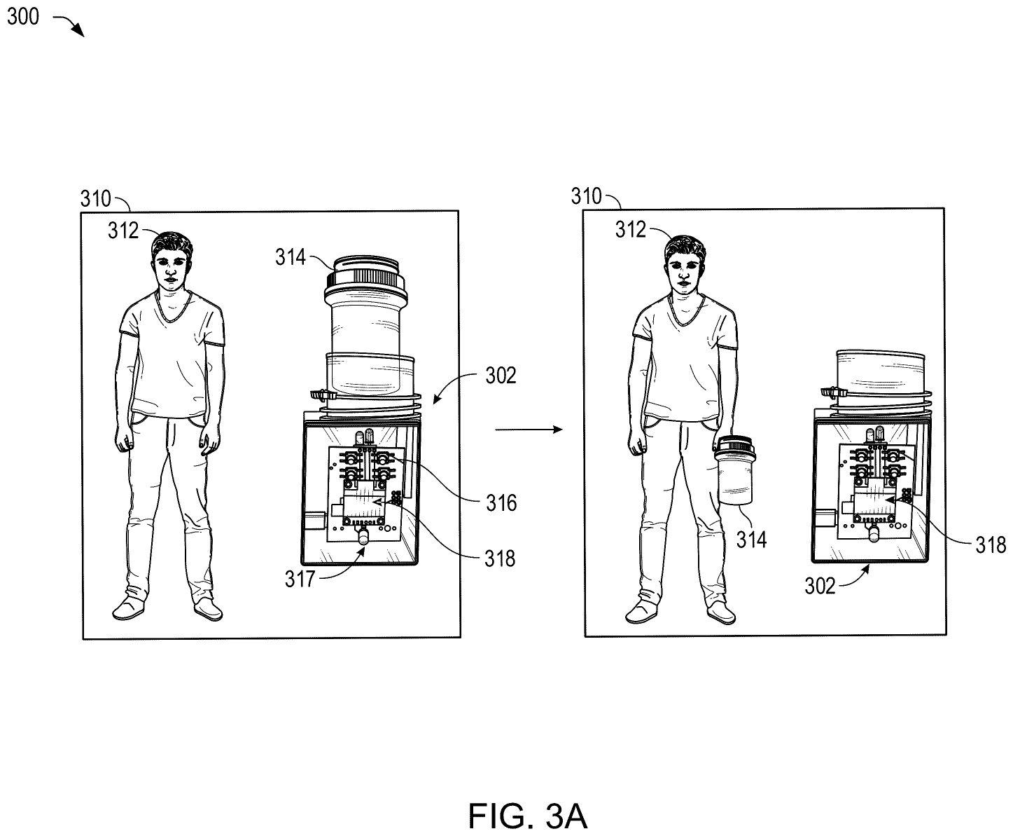

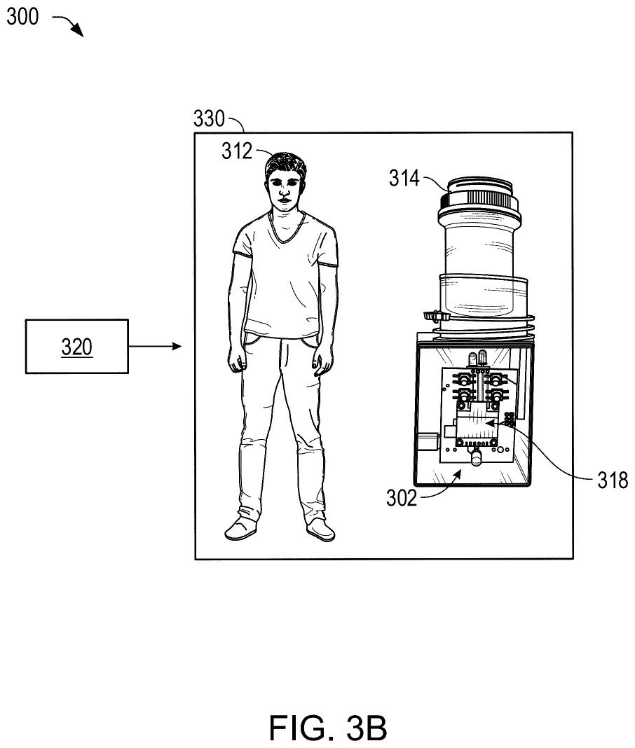

FIGS. 3A-3B illustrate an example of a use case 300 for managing medicine usage, in accordance with one or more example embodiments of the disclosure. The use case 300 illustrates example operations associated with usage of a medicine management apparatus 302 (which may be the same as, or similar to, medicine management apparatus 100 or any other medicine management apparatus described herein).

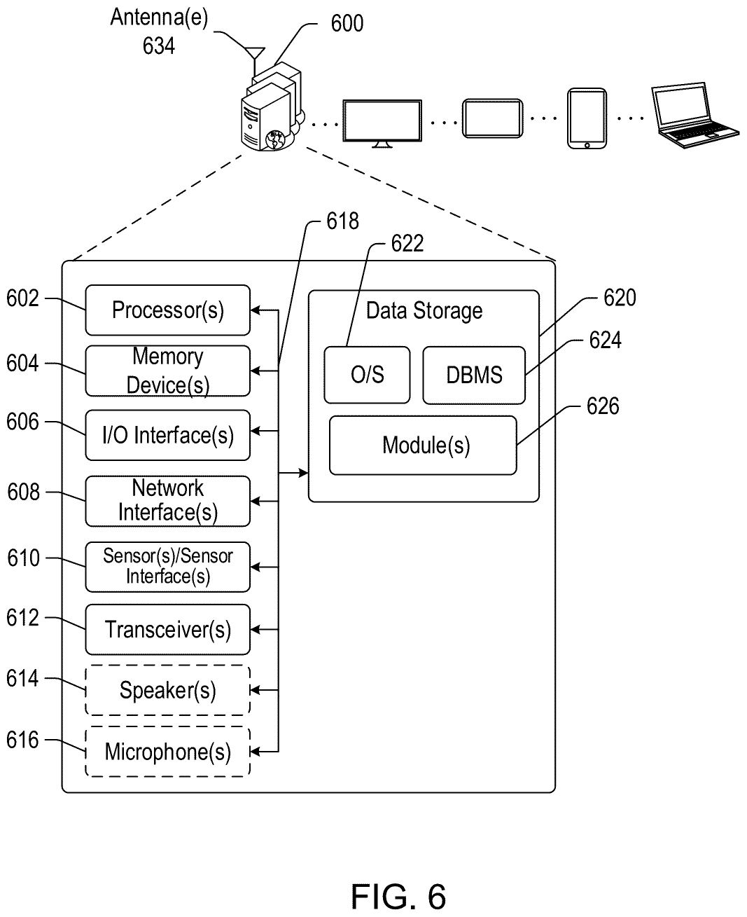

Beginning with scene 310, a user 312 is shown with the medicine management apparatus 302. The user 312 may be a person who is prescribed medicine included within medicine container 314 (such as user 602 shown in FIG. 6, for example). The medicine container 314 is initially provided on the medicine management apparatus 302. As shown in the scene 310, the medicine management apparatus 302 includes a controller 316 with an associated display 318. The display 318 is shown as presenting information about the contents of the medicine container 314 (a number of pills remaining in the medicine container 314). Particularly, the display 318 shows that five pills are remaining in the medicine container 314 at the time associated with the scene 310. An indication of the amount of medicine remaining in the medicine container 314 may also be presented in any other format (for example, number of remaining dosages, a number of days that the medicine in the medicine container 314 based on the dosages and number of remaining pills, etc.). As aforementioned, the display 316 may also present other types of information, such as alerts.

The scene 310 also shows that the medicine management apparatus 302 produces an alert that the user 312 should take a dosage of medicine from the medicine container 314. For example, the medicine management apparatus 302 includes a light emitting diode (LED) that may be illuminated by the controller 316 when the controller 316 determines that it is time for the user 312 to take a dosage of medicine. The alert may also be provided to the user 312 in any other form. For example, the medicine management apparatus 302 may include a speaker that may emit an auditory alert to the user, the alert may be presented via the display 318 as digital text, etc. The alert may also be transmitted to another device, such as a smartphone of the user 312, such that the user 312 may be alerted that they need to take a dosage of medicine even when they are not proximate to the medicine management apparatus 302. A similar type of alert may also be transmitted to a device of another user, such as a caretaker of the user 312, for example.

Turning to scenes 320 and 330, the user 312 is shown as removing the medicine container 314 from the medicine management apparatus 302 (scene 320) and returning the medicine container 314 to the medicine management apparatus 302 scene 33). For example, the user 312 may remove the medicine container 314 from the medicine management apparatus 302 to take a dosage of medicine from the medicine container 314. When the medicine container 314 is removed from the medicine management apparatus 302, the medicine management apparatus 302 senses that the medicine container 314 has been removed and updates the information about the amount of medication remaining in the medicine container 314. Specifically, the display 318 shows that the number of remaining pills is now four.

FIG. 4 depicts an example method 400 for training a machine learning model using compartmentalization. Some or all of the blocks of the process flows or methods in this disclosure may be performed in a distributed manner across any number of devices or systems. For example, the blocks may be performed by a controller of a medicine management apparatus (such as controller 104, for example). However, as mentioned below in FIG. 6, some or all of the processing may also be performed by other devices, such as user device(s), remote server(s), etc. The operations of the method 400 may be optional and may be performed in a different order.

At block 402 of the method 400, computer-executable instructions stored on a memory of a system or device, such as, controller 104, etc. may be executed to receive first data from one or more sensors of the apparatus, wherein the apparatus comprises a holding compartment configured to hold a medicine container and an enclosure housing a controller.

At block 404 of the method 400, computer-executable instructions stored on a memory of a system or device may be executed to determine, based on the first data, that the medicine container has been removed from the holding compartment and returned to the holding compartment.

At block 406 of the method 400, computer-executable instructions stored on a memory of a system or device may be executed to decrement a counter indicating a number of pills remaining in the medicine container.

At block 408 of the method 400, computer-executable instructions stored on a memory of a system or device may be executed to cause to present the number of pills remaining in the medicine container via a display of the apparatus.

FIG. 5 is an example system 500 for managing medicine usage. In one or more embodiments, the system may include one or more user devices (for example, user device 501, user device 503, and/or any other number of user devices), one or more computing devices 506, one or more medicine management apparatuses 508, and/or one or more databases 510. However, these components of the system 500 are merely exemplary and are not intended to be limiting in any way. For simplicity, reference may be made hereinafter to a user device 501, user device 503, computing device 506, medicine management apparatus 508, database 510, etc., however, this is not intended to be limiting and may still refer to any number of such elements.

The user device 501 and user device 503 may be any type of device, such as a smartphone, desktop computer, laptop computer, tablet, smart television (for example, a television with Internet connectivity, the capability to install applications, etc.), and/or any other type of device. The user device 501 may allow the user 502 to interact with any of the systems, devices, etc. to perform any number of different types of actions described herein. Likewise, the user device 503 may allow the user 504 to interact with any of the systems, devices, etc. to perform any number of different types of actions described herein.

The user 502 may be a person who is prescribed medicine that is being managed by the medicine management apparatus 508. As shown in FIG. 1, a medicine container 108 including the medicine may be provided on and/or in the medicine management apparatus 508. The user device 501 may include an application that allows the user 502 to view information produced by the medicine management apparatus 506. Any information that may be presented via the display of the medicine management apparatus 506 may also be transmitted to the user device 501 and presented to the user via a user interface of the application. That is, the application may supplement the medicine management apparatus 508 in assisting the user 502 in managing the usage of the medicine. For example, the application may present a number of pills remaining in the medicine container, a type of medicine included in the medicine container, information about usage of the medicine (frequency of usage, size of dosages, etc.), etc.

The application may also present any alerts to the user 502, such as an alert when the amount of medicine in the medicine container is less than a threshold amount of medicine (for example, a number of pills is less than a threshold number of pills, a volume of liquid is less than a threshold volume of liquid, etc.), an alert when it is time for the user 502 to take the medicine, etc. In instances when the medicine management apparatus 508 determines that the amount of medicine is less than the threshold amount of medicine, the application may provide an option for the user 502 (or the user 503) to place an order for a re-fill of the medication.

In some instances, the user 502 may have multiple medicine management apparatuses 508, each managing a different type of medication. Alternatively, the user 502 may have a single medicine management apparatus 508 that is capable of holding and managing multiple medicine containers at a single time. Accordingly, the application may allow the user 502 to track multiple medicine management apparatuses 508 and/or a single medicine management apparatus 508 that holds multiple medicine containers. That is, the application may allow the user to view information, receive alerts, etc. for any number of medicine management apparatuses 508 and/or medicine containers.

When multiple medicines are being tracked simultaneously, the application may also provide information about how different types of medicine interact. For example, the application may provide a warning to the user 502 via the application if two medicines counteract one another or if two types of medicines should not be taken together. This information may be obtained from the database 510 (or from any other source of data), for example.

It may also be desirable for this information to be trackable by a person (such as the user 504) other than the user 502 who is prescribed the medicine. For example, the user 502 may be an elderly patient and the user 504 may be a caretaker, family member, medical provider, or other type of third-party associated with the user 502. An application may also be provided on the user device 503 associated with the user 504 such that the user 504 may be able to view similar information and/or alerts that are viewable by the user 502 via the application of the user device 501.

The computing device 506 may be any type of device used to perform any of the processing described herein. For example, the computing device 506 may be a remote server that may supplement the medicine management apparatus 508 in some or all of the processing associated with managing medicine usage. However, the computing device 506 is also optional and all of the processing may also be performed locally by the medicine management apparatus 508.

The medicine management apparatus 508 may be the same as, or similar to the medicine management apparatus 100, medicine management apparatus 200, or any other medicine management apparatus described herein.

The database 510 may store information that is used by the medicine management apparatus 508 to track the usage of the medicine in the medicine container. For example, the database 510 may store indications of prescriptions associated with the user 502, dosage information for different types of medicine, a frequency at which the different types of medicine should be taken, a weight of each of the pills of medicine (to be used by the medicine management apparatus 508 to track whether a proper dosage was removed from the medicine container as aforementioned), and/or any other types of relevant information. Any of this information may also be stored in local memory of the medicine management apparatus 508 as well. The medicine management apparatus 508 may be in communication with the database 510 to obtain this information to manage the usage of the medicine in the medicine container as described herein.

In one or more embodiments, any of the elements of the system 500 (for example, one or more user devices, one or more computing devices 506, one or more medicine management apparatuses 508, and/or one or more databases 510, and/or any other element described with respect to FIG. 5 or otherwise) may be configured to communicate via a communications network 550. The communications network 550 may include, but not limited to, any one of a combination of different types of suitable communications networks such as, for example, broadcasting networks, cable networks, public networks (e.g., the Internet), private networks, wireless networks, cellular networks, or any other suitable private and/or public networks. Further, the communications network 550 may have any suitable communication range associated therewith and may include, for example, global networks (e.g., the Internet), metropolitan area networks (MANs), wide area networks (WANs), local area networks (LANs), or personal area networks (PANs). In addition, communications network 550 may include any type of medium over which network traffic may be carried including, but not limited to, coaxial cable, twisted-pair wire, optical fiber, a hybrid fiber coaxial (HFC) medium, microwave terrestrial transceivers, radio frequency communication mediums, white space communication mediums, ultra-high frequency communication mediums, satellite communication mediums, or any combination thereof.

Finally, any of the elements (for example, one or more user devices, one or more computing devices 506, one or more medicine management apparatuses 508, and/or one or more databases 510) of the system 500 may include any of the elements of the computing device 700 as well (such as the processor 702, memory 704, etc.).

FIG. 6 is a schematic block diagram of an illustrative computing device 600 in accordance with one or more example embodiments of the disclosure. The computing device 600 may include any suitable computing device capable of receiving and/or generating data including, but not limited to, a user device such as a smartphone, tablet, e-reader, wearable device, or the like; a desktop computer; a laptop computer; a content streaming device; a set-top box; or the like. The computing device 600 may correspond to an illustrative device configuration for the devices of FIGS. 1-5 (for example, medicine management apparatus 102, medicine management apparatus 302, users devices 501 and 503, computing device 506, etc.).

The computing device 600 may be configured to communicate via one or more networks with one or more servers, search engines, user devices, or the like. In some embodiments, a single remote server or single group of remote servers may be configured to perform more than one type of content rating and/or machine learning functionality.

Example network(s) may include, but are not limited to, any one or more different types of communications networks such as, for example, cable networks, public networks (e.g., the Internet), private networks (e.g., frame-relay networks), wireless networks, cellular networks, telephone networks (e.g., a public switched telephone network), or any other suitable private or public packet-switched or circuit-switched networks. Further, such network(s) may have any suitable communication range associated therewith and may include, for example, global networks (e.g., the Internet), metropolitan area networks (MANs), wide area networks (WANs), local area networks (LANs), or personal area networks (PANs). In addition, such network(s) may include communication links and associated networking devices (e.g., link-layer switches, routers, etc.) for transmitting network traffic over any suitable type of medium including, but not limited to, coaxial cable, twisted-pair wire (e.g., twisted-pair copper wire), optical fiber, a hybrid fiber-coaxial (HFC) medium, a microwave medium, a radio frequency communication medium, a satellite communication medium, or any combination thereof.

In an illustrative configuration, the computing device 600 may include one or more processors (processor(s)) 602, one or more memory devices 604 (generically referred to herein as memory 604), one or more input/output (I/O) interface(s) 606, one or more network interface(s) 608, one or more sensors or sensor interface(s) 610, one or more transceivers 612, one or more optional speakers 614, one or more optional microphones 616, and data storage 620. The computing device 600 may further include one or more buses 618 that functionally couple various components of the computing device 600. The computing device 600 may further include one or more antenna (e) 634 that may include, without limitation, a cellular antenna for transmitting or receiving signals to/from a cellular network infrastructure, an antenna for transmitting or receiving Wi-Fi signals to/from an access point (AP), a Global Navigation Satellite System (GNSS) antenna for receiving GNSS signals from a GNSS satellite, a Bluetooth antenna for transmitting or receiving Bluetooth signals, a Near Field Communication (NFC) antenna for transmitting or receiving NFC signals, and so forth. These various components will be described in more detail hereinafter.

The bus(es) 618 may include at least one of a system bus, a memory bus, an address bus, or a message bus, and may permit exchange of information (e.g., data (including computer-executable code), signaling, etc.) between various components of the computing device 600. The bus(es) 618 may include, without limitation, a memory bus or a memory controller, a peripheral bus, an accelerated graphics port, and so forth. The bus(es) 618 may be associated with any suitable bus architecture including, without limitation, an Industry Standard Architecture (ISA), a Micro Channel Architecture (MCA), an Enhanced ISA (EISA), a Video Electronics Standards Association (VESA) architecture, an Accelerated Graphics Port (AGP) architecture, a Peripheral Component Interconnects (PCI) architecture, a PCI-Express architecture, a Personal Computer Memory Card International Association (PCMCIA) architecture, a Universal Serial Bus (USB) architecture, and so forth.

The memory 604 of the computing device 600 may include volatile memory (memory that maintains its state when supplied with power) such as random access memory (RAM) and/or non-volatile memory (memory that maintains its state even when not supplied with power) such as read-only memory (ROM), flash memory, ferroelectric RAM (FRAM), and so forth. Persistent data storage, as that term is used herein, may include non-volatile memory. In certain example embodiments, volatile memory may enable faster read/write access than non-volatile memory. However, in certain other example embodiments, certain types of non-volatile memory (e.g., FRAM) may enable faster read/write access than certain types of volatile memory.

In various implementations, the memory 604 may include multiple different types of memory such as various types of static random access memory (SRAM), various types of dynamic random access memory (DRAM), various types of unalterable ROM, and/or writeable variants of ROM such as electrically erasable programmable read-only memory (EEPROM), flash memory, and so forth. The memory 604 may include main memory as well as various forms of cache memory such as instruction cache(s), data cache(s), translation lookaside buffer(s) (TLBs), and so forth. Further, cache memory such as a data cache may be a multi-level cache organized as a hierarchy of one or more cache levels (L1, L2, etc.).

The data storage 620 may include removable storage and/or non-removable storage including, but not limited to, magnetic storage, optical disk storage, and/or tape storage. The data storage 620 may provide non-volatile storage of computer-executable instructions and other data. The memory 604 and the data storage 620, removable and/or non-removable, are examples of computer-readable storage media (CRSM) as that term is used herein.

The data storage 620 may store computer-executable code, instructions, or the like that may be loadable into the memory 604 and executable by the processor(s) 602 to cause the processor(s) 602 to perform or initiate various operations. The data storage 620 may additionally store data that may be copied to memory 604 for use by the processor(s) 602 during the execution of the computer-executable instructions. Moreover, output data generated as a result of execution of the computer-executable instructions by the processor(s) 602 may be stored initially in memory 604, and may ultimately be copied to data storage 620 for non-volatile storage.

More specifically, the data storage 620 may store one or more operating systems (O/S) 622; one or more database management systems (DBMS) 624; and one or more program module(s), applications, engines, computer-executable code, scripts, or the like such as, for example, one or more module(s) 626. Any of the components depicted as being stored in data storage 620 may include any combination of software, firmware, and/or hardware. The software and/or firmware may include computer-executable code, instructions, or the like that may be loaded into the memory 604 for execution by one or more of the processor(s) 602. Any of the components depicted as being stored in data storage 620 may support functionality described in reference to correspondingly named components earlier in this disclosure.

The data storage 620 may further store various types of data utilized by components of the computing device 600. Any data storaged in the data storage 620 may be loaded into the memory 604 for use by the processor(s) 602 in executing computer-executable code. In addition, any data depicted as being stored in the data storage 620 may potentially be stored in one or more datastore(s) and may be accessed via the DBMS 624 and loaded in the memory 604 for use by the processor(s) 602 in executing computer-executable code. The datastore(s) may include, but are not limited to, databases (e.g., relational, object-oriented, etc.), file systems, flat files, distributed datastores in which data is stored on more than one node of a computer network, peer-to-peer network datastores, or the like. In FIG. 6, the datastore(s) may include, for example, purchase history information, user action information, user profile information, a database linking search queries and user actions, and other information.

The processor(s) 602 may be configured to access the memory 604 and execute computer-executable instructions loaded therein. For example, the processor(s) 602 may be configured to execute computer-executable instructions of the various program module(s), applications, engines, or the like of the computing device 600 to cause or facilitate various operations to be performed in accordance with one or more embodiments of the disclosure. The processor(s) 602 may include any suitable processing unit capable of accepting data as input, processing the input data in accordance with stored computer-executable instructions, and generating output data. The processor(s) 602 may include any type of suitable processing unit including, but not limited to, a central processing unit, a microprocessor, a Reduced Instruction Set Computer (RISC) microprocessor, a Complex Instruction Set Computer (CISC) microprocessor, a microcontroller, an Application Specific Integrated Circuit (ASIC), a Field-Programmable Gate Array (FPGA), a System-on-a-Chip (SoC), a digital signal processor (DSP), and so forth. Further, the processor(s) 602 may have any suitable microarchitecture design that includes any number of constituent components such as, for example, registers, multiplexers, arithmetic logic units, cache controllers for controlling read/write operations to cache memory, branch predictors, or the like. The microarchitecture design of the processor(s) 602 may be capable of supporting any of a variety of instruction sets.

Referring now to functionality supported by the various program module(s) depicted in FIG. 6, the module(s) 626 may include computer-executable instructions, code, or the like that responsive to execution by one or more of the processor(s) 602 may perform any functions of any of the systems and/or devices described herein (e.g., a UAV, user device, computing device, docking station, groundwater collection and analysis apparatus, etc.

Referring now to other illustrative components depicted as being stored in the data storage 620, the O/S 622 may be loaded from the data storage 620 into the memory 604 and may provide an interface between other application software executing on the computing device 600 and hardware resources of the computing device 600. More specifically, the O/S 622 may include a set of computer-executable instructions for managing hardware resources of the computing device 600 and for providing common services to other application programs (e.g., managing memory allocation among various application programs). In certain example embodiments, the O/S 622 may control execution of the other program module(s) to dynamically enhance characters for content rendering. The O/S 622 may include any operating system now known or which may be developed in the future including, but not limited to, any server operating system, any mainframe operating system, or any other proprietary or non-proprietary operating system.

The DBMS 624 may be loaded into the memory 604 and may support functionality for accessing, retrieving, storing, and/or manipulating data storaged in the memory 604 and/or data storaged in the data storage 620. The DBMS 624 may use any of a variety of database models (e.g., relational model, object model, etc.) and may support any of a variety of query languages. The DBMS 624 may access data represented in one or more data schemas and stored in any suitable data repository including, but not limited to, databases (e.g., relational, object-oriented, etc.), file systems, flat files, distributed datastores in which data is stored on more than one node of a computer network, peer-to-peer network datastores, or the like. In those example embodiments in which the computing device 600 is a user device, the DBMS 624 may be any suitable light-weight DBMS optimized for performance on a user device.

Referring now to other illustrative components of the computing device 600, the input/output (I/O) interface(s) 606 may facilitate the receipt of input information by the computing device 600 from one or more I/O devices as well as the output of information from the computing device 600 to the one or more I/O devices. The I/O devices may include any of a variety of components such as a display or display screen having a touch surface or touchscreen; an audio output device for producing sound, such as a speaker; an audio capture device, such as a microphone; an image and/or video capture device, such as a camera; a haptic unit; and so forth. Any of these components may be integrated into the computing device 600 or may be separate. The I/O devices may further include, for example, any number of peripheral devices such as data storage devices, printing devices, and so forth.

The I/O interface(s) 606 may also include an interface for an external peripheral device connection such as universal serial bus (USB), FireWire, Thunderbolt, Ethernet port or other connection protocol that may connect to one or more networks. The I/O interface(s) 606 may also include a connection to one or more of the antenna (e) 634 to connect to one or more networks via a wireless local area network (WLAN) (such as Wi-Fi) radio, Bluetooth, ZigBee, and/or a wireless network radio, such as a radio capable of communication with a wireless communication network such as a Long Term Evolution (LTE) network, WiMAX network, 3G network, ZigBee network, etc.

The computing device 600 may further include one or more network interface(s) 608 via which the computing device 600 may communicate with any of a variety of other systems, platforms, networks, devices, and so forth. The network interface(s) 608 may enable communication, for example, with one or more wireless routers, one or more host servers, one or more web servers, and the like via one or more of networks.

The antenna (e) 634 may include any suitable type of antenna depending, for example, on the communications protocols used to transmit or receive signals via the antenna (e) 634. Non-limiting examples of suitable antennas may include directional antennas, non-directional antennas, dipole antennas, folded dipole antennas, patch antennas, multiple-input multiple-output (MIMO) antennas, or the like. The antenna (e) 634 may be communicatively coupled to one or more transceivers 612 or radio components to which or from which signals may be transmitted or received.

As previously described, the antenna (e) 634 may include a cellular antenna configured to transmit or receive signals in accordance with established standards and protocols, such as Global System for Mobile Communications (GSM), 3G standards (e.g., Universal Mobile Telecommunications System (UMTS), Wideband Code Division Multiple Access (W-CDMA), CDMA2000, etc.), 4G standards (e.g., Long-Term Evolution (LTE), WiMax, etc.), direct satellite communications, or the like.

The antenna (e) 634 may additionally, or alternatively, include a Wi-Fi antenna configured to transmit or receive signals in accordance with established standards and protocols, such as the IEEE 802.11 family of standards, including via 2.4 GHz channels (e.g., 802.11b, 802.11g, 802.11n), 5 GHz channels (e.g., 802.11n, 802.11ac), or 60 GHz channels (e.g., 802.11ad). In alternative example embodiments, the antenna (e) 634 may be configured to transmit or receive radio frequency signals within any suitable frequency range forming part of the unlicensed portion of the radio spectrum.

The antenna (e) 634 may additionally, or alternatively, include a GNSS antenna configured to receive GNSS signals from three or more GNSS satellites carrying time-position information to triangulate a position therefrom. Such a GNSS antenna may be configured to receive GNSS signals from any current or planned GNSS such as, for example, the Global Positioning System (GPS), the GLONASS System, the Compass Navigation System, the Galileo System, or the Indian Regional Navigational System.

The transceiver(s) 612 may include any suitable radio component(s) for—in cooperation with the antenna (e) 634—transmitting or receiving radio frequency (RF) signals in the bandwidth and/or channels corresponding to the communications protocols utilized by the computing device 600 to communicate with other devices. The transceiver(s) 612 may include hardware, software, and/or firmware for modulating, transmitting, or receiving—potentially in cooperation with any of antenna (e) 634—communications signals according to any of the communications protocols discussed above including, but not limited to, one or more Wi-Fi and/or Wi-Fi direct protocols, as standardized by the IEEE 802.11 standards, one or more non-Wi-Fi protocols, or one or more cellular communications protocols or standards. The transceiver(s) 612 may further include hardware, firmware, or software for receiving GNSS signals. The transceiver(s) 612 may include any known receiver and baseband suitable for communicating via the communications protocols utilized by the computing device 600. The transceiver(s) 612 may further include a low noise amplifier (LNA), additional signal amplifiers, an analog-to-digital (A/D) converter, one or more buffers, a digital baseband, or the like.

The sensor(s)/sensor interface(s) 610 may include or may be capable of interfacing with any suitable type of sensing device such as, for example, inertial sensors, force sensors, thermal sensors, and so forth. Example types of inertial sensors may include accelerometers (e.g., MEMS-based accelerometers), gyroscopes, and so forth.

The optional speaker(s) 614 may be any device configured to generate audible sound. The optional microphone(s) 616 may be any device configured to receive analog sound input or voice data.

It should be appreciated that the program module(s), applications, computer-executable instructions, code, or the like depicted in FIG. 6 as being stored in the data storage 620 are merely illustrative and not exhaustive and that processing described as being supported by any particular module may alternatively be distributed across multiple module(s) or performed by a different module. In addition, various program module(s), script(s), plug-in(s), Application Programming Interface(s) (API(s)), or any other suitable computer-executable code hosted locally on the computing device 600, and/or hosted on other computing device(s) accessible via one or more networks, may be provided to support functionality provided by the program module(s), applications, or computer-executable code depicted in FIG. 6 and/or additional or alternate functionality. Further, functionality may be modularized differently such that processing described as being supported collectively by the collection of program module(s) depicted in FIG. 6 may be performed by a fewer or greater number of module(s), or functionality described as being supported by any particular module may be supported, at least in part, by another module. In addition, program module(s) that support the functionality described herein may form part of one or more applications executable across any number of systems or devices in accordance with any suitable computing model such as, for example, a client-server model, a peer-to-peer model, and so forth. In addition, any of the functionality described as being supported by any of the program module(s) depicted in FIG. 6 may be implemented, at least partially, in hardware and/or firmware across any number of devices.

It should further be appreciated that the computing device 600 may include alternate and/or additional hardware, software, or firmware components beyond those described or depicted without departing from the scope of the disclosure. More particularly, it should be appreciated that software, firmware, or hardware components depicted as forming part of the computing device 600 are merely illustrative and that some components may not be present or additional components may be provided in various embodiments. While various illustrative program module(s) have been depicted and described as software module(s) stored in data storage 620, it should be appreciated that functionality described as being supported by the program module(s) may be enabled by any combination of hardware, software, and/or firmware. It should further be appreciated that each of the above-mentioned module(s) may, in various embodiments, represent a logical partitioning of supported functionality. This logical partitioning is depicted for ease of explanation of the functionality and may not be representative of the structure of software, hardware, and/or firmware for implementing the functionality. Accordingly, it should be appreciated that functionality described as being provided by a particular module may, in various embodiments, be provided at least in part by one or more other module(s). Further, one or more depicted module(s) may not be present in certain embodiments, while in other embodiments, additional module(s) not depicted may be present and may support at least a portion of the described functionality and/or additional functionality. Moreover, while certain module(s) may be depicted and described as sub-module(s) of another module, in certain embodiments, such module(s) may be provided as independent module(s) or as sub-module(s) of other module(s).

Program module(s), applications, or the like disclosed herein may include one or more software components including, for example, software objects, methods, data structures, or the like. Each such software component may include computer-executable instructions that, responsive to execution, cause at least a portion of the functionality described herein (e.g., one or more operations of the illustrative methods described herein) to be performed.

A software component may be coded in any of a variety of programming languages. An illustrative programming language may be a lower-level programming language such as an assembly language associated with a particular hardware architecture and/or operating system platform. A software component comprising assembly language instructions may require conversion into executable machine code by an assembler prior to execution by the hardware architecture and/or platform.

Another example programming language may be a higher-level programming language that may be portable across multiple architectures. A software component comprising higher-level programming language instructions may require conversion to an intermediate representation by an interpreter or a compiler prior to execution.

Other examples of programming languages include, but are not limited to, a macro language, a shell or command language, a job control language, a script language, a database query or search language, or a report writing language. In one or more example embodiments, a software component comprising instructions in one of the foregoing examples of programming languages may be executed directly by an operating system or other software component without having to be first transformed into another form.

A software component may be stored as a file or other data storage construct. Software components of a similar type or functionally related may be stored together such as, for example, in a particular directory, folder, or library. Software components may be static (e.g., pre-established or fixed) or dynamic (e.g., created or modified at the time of execution).

Software components may invoke or be invoked by other software components through any of a wide variety of mechanisms. Invoked or invoking software components may comprise other custom-developed application software, operating system functionality (e.g., device drivers, data storage (e.g., file management) routines, other common routines and services, etc.), or third-party software components (e.g., middleware, encryption, or other security software, database management software, file transfer or other network communication software, mathematical or statistical software, image processing software, and format translation software).

Software components associated with a particular solution or system may reside and be executed on a single platform or may be distributed across multiple platforms. The multiple platforms may be associated with more than one hardware vendor, underlying chip technology, or operating system. Furthermore, software components associated with a particular solution or system may be initially written in one or more programming languages, but may invoke software components written in another programming language.

Computer-executable program instructions may be loaded onto a special-purpose computer or other particular machine, a processor, or other programmable data processing apparatus to produce a particular machine, such that execution of the instructions on the computer, processor, or other programmable data processing apparatus causes one or more functions or operations specified in the flow diagrams to be performed. These computer program instructions may also be stored in a computer-readable storage medium (CRSM) that upon execution may direct a computer or other programmable data processing apparatus to function in a particular manner, such that the instructions stored in the computer-readable storage medium produce an article of manufacture including instruction means that implement one or more functions or operations specified in the flow diagrams. The computer program instructions may also be loaded onto a computer or other programmable data processing apparatus to cause a series of operational elements or steps to be performed on the computer or other programmable apparatus to produce a computer-implemented process.

Additional types of CRSM that may be present in any of the devices described herein may include, but are not limited to, programmable random access memory (PRAM), SRAM, DRAM, RAM, ROM, electrically erasable programmable read-only memory (EEPROM), flash memory or other memory technology, compact disc read-only memory (CD-ROM), digital versatile disc (DVD) or other optical storage, magnetic cassettes, magnetic tape, magnetic disk storage or other magnetic storage devices, or any other medium which can be used to store the information and which can be accessed. Combinations of any of the above are also included within the scope of CRSM. Alternatively, computer-readable communication media (CRCM) may include computer-readable instructions, program module(s), or other data transmitted within a data signal, such as a carrier wave, or other transmission. However, as used herein, CRSM does not include CRCM.

Although embodiments have been described in language specific to structural features and/or methodological acts, it is to be understood that the disclosure is not necessarily limited to the specific features or acts described. Rather, the specific features and acts are disclosed as illustrative forms of implementing the embodiments. Conditional language, such as, among others, “can,” “could,” “might,” or “may,” unless specifically stated otherwise, or otherwise understood within the context as used, is generally intended to convey that certain embodiments could include, while other embodiments do not include, certain features, elements, and/or steps. Thus, such conditional language is not generally intended to imply that features, elements, and/or steps are in any way required for one or more embodiments or that one or more embodiments necessarily include logic for deciding, with or without user input or prompting. whether these features. elements, and/or steps are included or are to be performed in any particular embodiment.

That which is claimed is:

1. An apparatus comprising:
a holding compartment configured to hold a medicine container;
one or more sensors; and
an enclosure housing a controller, wherein the controller is configured to:
receive first data from the one or more sensors;
determine, based on the first data, that the medicine container has been removed from the holding compartment and returned to the holding compartment; and
decrement a counter indicating a number of pills remaining in the medicine container; and
cause to present the number of pills remaining in the medicine container via a display.

2. The apparatus of claim 1, wherein the one or more sensors include at least one of: one or more pressure sensors or one or more proximity sensors.

3. The apparatus of claim 1, wherein the one or more sensors include one or more optical sensors.

4. The apparatus of claim 1, wherein the controller is further configured to:
receive second data from the one or more sensors;
determine a difference between the first data and the second data;
determine, based on the difference that an improper dosage of medicine was removed from the medicine container; and
causing to provide an alert.

5. The apparatus of claim 1, wherein the controller is further configured to:
determine that a threshold amount of time has passed; and
cause to provide an alert indicating that a dosage of medication from the medicine container should be taken.

6. The apparatus of claim 5, wherein the alert is at least one of: an auditory alert or a visual alert.

7. The apparatus of claim 5, wherein the alert is transmitted to a separate device.

8. The apparatus of claim 1, wherein the controller is further configured to:
determine that the number of pills remaining in the medicine container is less than a threshold number; and
cause to provide an alert indicating that the medicine container should be re-filled.

9. A method comprising:
receiving, by a controller of an apparatus, first data from one or more sensors of the apparatus, wherein the apparatus comprises a holding compartment configured to hold a medicine container and an enclosure housing a controller;
determining, by the controller and based on the first data, that the medicine container has been removed from the holding compartment and returned to the holding compartment;
decrementing, by the controller, a counter indicating a number of pills remaining in the medicine container; and
causing, by the controller, to present the number of pills remaining in the medicine container via a display of the apparatus.

10. The method of claim 9, further comprising:
receiving, by the controller, second data from the one or more sensors;
determining, by the controller, a difference between the first data and the second data;
determining, by the controller and based on the difference, that an improper dosage of medicine was removed from the medicine container; and
causing, by the controller, to provide an alert.

11. The method of claim 9, further comprising:
determining, by the controller, that a threshold amount of time has passed; and
causing, by the controller, to provide an alert indicating that a dosage of medication from the medicine container should be taken.

12. The method of claim 11, wherein the alert is at least one of: an auditory alert or a visual alert.

13. The method of claim 11, wherein the alert is transmitted to a separate device.

14. The method of claim 9, further comprising:
determining, by the controller, that the number of pills remaining in the medicine container is less than a threshold number; and
causing, by the controller, to provide an alert indicating that the medicine container should be re-filled.

15. A system comprising:
a holding compartment configured to hold a medicine container;
one or more sensors; and
an enclosure housing a controller, wherein the controller is configured to:
receive first data from the one or more sensors;
determine, based on the first data, that the medicine container has been removed from the holding compartment and returned to the holding compartment; and
decrement a counter indicating a number of pills remaining in the medicine container; and
cause to present the number of pills remaining in the medicine container via a display.

16. The system of claim 15, wherein the one or more sensors include one or more pressure sensors.

17. The system of claim 15, wherein the one or more sensors include one or more optical sensors.

18. The system of claim 15, wherein the controller is further configured to:
receive second data from the one or more sensors;
determine a difference between the first data and the second data;
determine, based on the difference that an improper dosage of medicine was removed from the medicine container; and
causing to provide an alert.

19. The system of claim 15, wherein the controller is further configured to:
determine that a threshold amount of time has passed; and
cause to provide an alert indicating that a dosage of medication from the medicine container should be taken.

20. The system of claim 19, wherein the alert is at least one of: an auditory alert or a visual alert.
